(19) United States
(12) Patent Application Publication
(10) Pub. No.: US 2009/0124506 A1
(43) Pub. Date: May 14, 2009

(54) Membrane computing

(75) Inventor: Ehud Keinan, Timrat (IL)

Correspondence Address: MARTIN D. MOYNIHAN d/b/a PRTSI, INC., P.O. BOX 16446, ARLINGTON, VA, 22215, US

(73) Assignee: Technion Research & Development Foundation Ltd., Haifa (IL)

(21) Appl. No.: 12/289,735

(22) Filed: Nov. 3, 2008

Related U.S. Application Data
(60) Provisional application No. 60/984,834, filed on Nov. 2, 2007.

(51) Int. Cl. C40B 30/00; B01J 19/00; C12M 1/00; G01N 21/64; G01N 21/76; C12Q 1/00

(52) U.S. Cl. 506/7; 422/68.1; 435/287.1; 435/287.2; 422/82.08; 422/52; 435/4

(57) ABSTRACT

A method of implementation of a P-system in membrane computing comprising: placing three mutually immiscible liquids into a container, such that a first of said liquids provides a barrier between the second and third liquids; providing an initial substrate to at least one of said second and third liquids; and applying a transport rule for said first liquid to allow transport of substrate between said second and said third liquid.

### RELATED APPLICATION/S

This application claims the benefit of U.S. Provisional Patent Application No. 60/984,834 filed Nov. 2, 2007.

The contents of the above document is incorporated by reference as if fully set forth herein.

### FIELD AND BACKGROUND OF THE INVENTION

The present invention, in some embodiments thereof, relates to an apparatus and method for carrying out membrane computing in practice and, more particularly, but not exclusively, to an apparatus and method of providing a practical membrane having the properties required by membrane computing to separate between two phases while allowing controlled functional interaction between them.

Membrane computing (MC) is an area of computer science aiming to abstract computing ideas and models from the structure and the functioning of living cells, as well as from the way the cells are organized in tissues or higher order structures. In short, MC deals with distributed and parallel computing models, processing multisets of symbol objects in a localized manner. Thus, evolution rules and evolving objects are encapsulated into compartments defined by membranes. The communications between compartments and with the environment play an essential role in the processes. The various types of membrane systems are known as P systems after Gheorghe Paun who first conceived the model in 1998.

An essential ingredient of a P system is its membrane structure, which can be a hierarchical arrangement of membranes, as in a cell, or a net of membranes (placed in the nodes of a graph), as in a tissue or a neural net. P systems are often depicted graphically with drawings such as that shown in FIG. 1. The intuition behind the notion of a membrane is a three-dimensional vesicle from biology, but the concept itself is generalized or idealized to interpreting a membrane as a separator of two regions, providing the possibility of selective communication between the two regions. As per Gheorghe Paun, the separation is of the Euclidean space into a finite “inside” and an infinite “outside”.

Graphical representations may have other elements according to the variation of the model that is being studied. For example, a rule may produce the special symbol δ, in which case the membrane that contains it is dissolved and all its contents move up in the region hierarchy.

The variety of suggestions from biology and the range of possibilities to define the architecture and the functioning of a membrane-based multiset processing device are practically endless, and already the literature of MC contains a very large number of models. Thus, MC is not merely a theory related to a specific model, it is a framework for devising compartmentalized models.

Chemicals are modeled by symbols, or alternatively by strings of symbols. The region, which is defined by a membrane, can contain other symbols or strings (collectively referred to as objects) or other membranes, so that a P system has exactly one outer membrane, called the skin membrane, and a hierarchical relationship governing all its membranes under the skin membrane.

If objects are symbols, then their multiplicity within a region matters; however multi-sets are also used in some string models. Regions have associated rules that define how objects are produced, consumed, passed to other regions and otherwise interact with one another. The nondeterministic maximally parallel application of rules throughout the system is a transition between system states, and a sequence of transitions is called a computation. Particular goals can be defined to signify a halting state, at which point the result of the computation would be the objects contained in a particular region. Alternatively the result may be made up of objects sent out of the skin membrane to the environment.

Many variant models have been studied, and the main interest has been focused on proving computational universality for systems with a small number of membranes, for the purpose of solving NP-complete problems such as Boolean satisfiabilty (SAT) problems and the traveling salesman problem (TSP). The P systems may trade space and time complexities and less often use models to explain natural processes in living cells. The studies devise models that may at least theoretically be implemented on hardware. However, to date, the P systems are all theoretical models that have never been reduced to practice.

### SUMMARY OF THE INVENTION

Herein, we propose a general strategy to construct laboratory scale P systems that can be practically used for membrane computing. This technology is based on modular arrays of interconnected liquid membrane devices.

According to an aspect of some embodiments of the present invention there is provided a component for computing comprising:

a first liquid phase,

a liquid separation, and

a second liquid phase separated from the first liquid phase by the liquid separation, at least one of the liquid phases initially comprising a reaction substrate and a reaction rule and the liquid separation comprising a transport rule, such as to allow reactions and transportation to occur in the component, a computing result being obtained from at least one of substrate identification and location following the reactions and transportation.

In an embodiment, the liquid and separation phases are retained by mutual immiscibility of the respective liquids.

In an embodiment, the transport rule is provided by a phase transfer catalyst.

In an embodiment, the reaction rule is provided by one member of the group comprising an enzyme, a mixture of enzymes, a biocatalyst, a catalytic antibody, an organic catalyst, an organometallic catalyst, and a reagent.

In an embodiment, transport is assisted by one member of the group comprising a mixing device located within the liquid separation, photochemical activity and electrochemical activity.

In an embodiment, the liquid component comprises a transport mechanism being any one of the group comprising cation transport, anion transport, neutral molecule transport and switchable transport.

In an embodiment, detection of the computing result comprises making use of any one of the group comprising: change of color, colorimetry, pH, fluorescence, electrochemically active compounds, chemoluminescence, change in ion concentration, change in Ph, and change in conductivity.

An embodiment may include branches for connecting each liquid phase to that of a neighboring component, thereby to allow construction of a component array.

According to a second aspect of the present invention there is provided a method of implementation of a P-system in membrane computing comprising:

placing three mutually immiscible liquids into a container, such that a first of the liquids provides a barrier between the second and third liquids;

providing an initial substrate to at least one of the second and third liquids; and

applying a transport rule for the first liquid to allow transport of substrate between the second and the third liquid.

The method may comprise providing the transport rule by insertion of a phase transfer catalyst into the first liquid.

The method may comprise providing a reaction rule in at least one of the second and third liquids.

The method may comprise providing the reaction rule by addition of at least one member of the group comprising an enzyme, a mixture of enzymes, a biocatalyst, a catalytic antibody, an organic catalyst, an organometallic catalyst, and a reagent, to the at least one liquid, the member selected to catalyze a reaction on a substrate.

The method may comprise adding substrates, phase transfer catalysts and enzymes to the respective liquids to provide initial conditions for a predetermined computing function.

The method may comprise combining a plurality of the containers into an array, such that the liquids define a plurality of reaction regions separated by a plurality of barriers.

In the method, one of the barriers may provide symport transport, or one of the barriers may provide antiport transport.

In the method, the second and third liquids may be aqueous, the first liquid may then be non-aqueous, and a substrate is hydrophobic.

Alternatively, the second and third liquids may be non-aqueous, the first liquid may be aqueous, and a substrate may then be hydrophillic.

The method may comprise detecting a computing result by making use of any one of the group comprising: change of color, colorimetry, pH, fluorescence, electrochemically active compounds, chemoluminescence, change in ion concentration, for example change in Ph, and change in conductivity.

In a variation, wherein the second and third liquids are aqueous, and the first liquid is non-aqueous, it may be that the phase transfer catalyst is hydrophobic.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified diagram illustrating a classic P-system in membrane computing, until now unrealized in practice;

FIG. 2 is a simplified diagram illustrating a practical implementation of a membrane computing component in which two phases are separated by a membrane according to a first embodiment of the present invention;

FIG. 3 is a simplified diagram illustrating an alternative to FIG. 2 in which the phases are connectable to multiple neighboring phases, allowing a complex array to be constructed;

FIG. 4 is a simplified diagram illustrating a variation of the component of FIG. 2 in which the membrane is lighter than the aqueous phases;

FIG. 5A is a simplified diagram showing a membrane according to the present embodiments through which symport transportation occurs;

FIG. 5B is a simplified diagram showing a membrane according to the present embodiments through which antiport transportation occurs;

FIG. 6 is a simplified diagram showing an implementation of the P-system of FIG. 1 using an array of three U-tubes;

FIG. 7 is a schematic diagram illustrating a P-system incorporating nine regions;

FIG. 8 shows the P-system of FIG. 8 illustrated as a branch diagram; and

FIG. 9 is a practical implementation using the present embodiments in which U-tubes containing liquids provide components of the P-system of FIG. 7.

### DESCRIPTION OF EMBODIMENTS OF THE INVENTION

The present invention, in some embodiments thereof, relates to an apparatus and method for carrying out membrane computing in practice and, more particularly, but not exclusively, to an apparatus and method of providing a practical membrane having the properties required by membrane computing to separate between two phases while allowing controlled functional interaction between them.

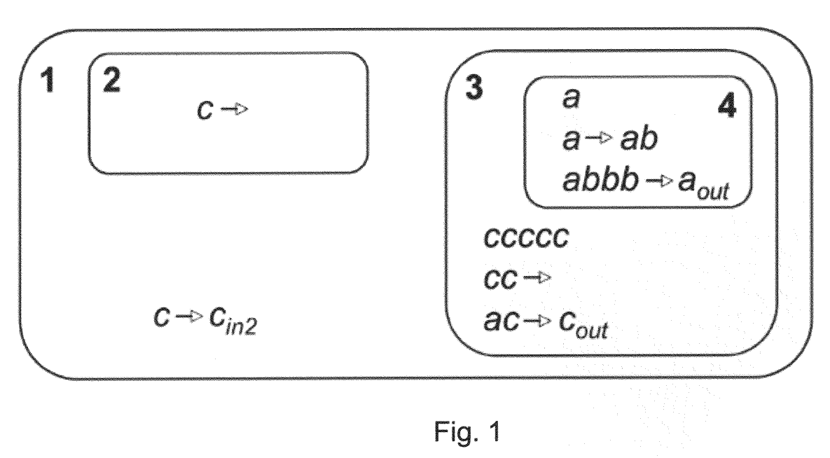

For purposes of better understanding some embodiments of the present invention, reference is first made to the theoretical P model as illustrated in FIG. 1. It is noted that since this model has never been reduced to practice to date it is not believed to be prior art in the patent sense even though it has been previously published.

FIG. 1 illustrates an example of a P system, currently only known from theory, and represented graphically with four membranes labeled with consecutive integers. Membrane 1 is the skin membrane, and contains membranes two and three as well as a rule. The particular rule c→cᵢₙ₂, moves any symbol c it encounters into region 2. This is possible because region 2 is directly accessible to region 1.

Region 2 in turn contains a rule c→, that destroys c symbols. Region 3 initially contains five c symbols and two rules. The first rule cc→, takes two c symbols and erases them as before, so that after 2 transitions only one symbol will remain. A second rule ac→cₒᵤₜ, takes an a and a c symbol and destroys the a symbol while delivering the c symbol to region 1.

Region 4 is essentially a counter. After three transitions the a symbol is sent out of the membrane into region 3. The second rule of region 3 consumes the a symbol and the one remaining c symbol and sends a c symbol out to region 1. The symbol is eventually destroyed in region 2.

The following embodiments teach how a system according to FIG. 1 can be implemented on a practical apparatus. Before explaining at least one embodiment of the invention in detail, it is to be understood that the invention is not necessarily limited in its application to the details of construction and the arrangement of the components and/or methods set forth in the following description and/or illustrated in is the drawings and/or the Examples. The invention is capable of other embodiments or of being practiced or carried out in various ways.

In implementing a P-system a first challenge is to create a practical membrane that allows for a membrane within a membrane structure to emerge. A second challenge is that the membrane within a membrane structure should be stable so that a particular region retains the same neighbors for the duration of the exercise. If for example an inner membrane were to bounce around so its neighbors change several times then the computing structure becomes undefined. Thirdly the membrane structures should have controllable transport properties.

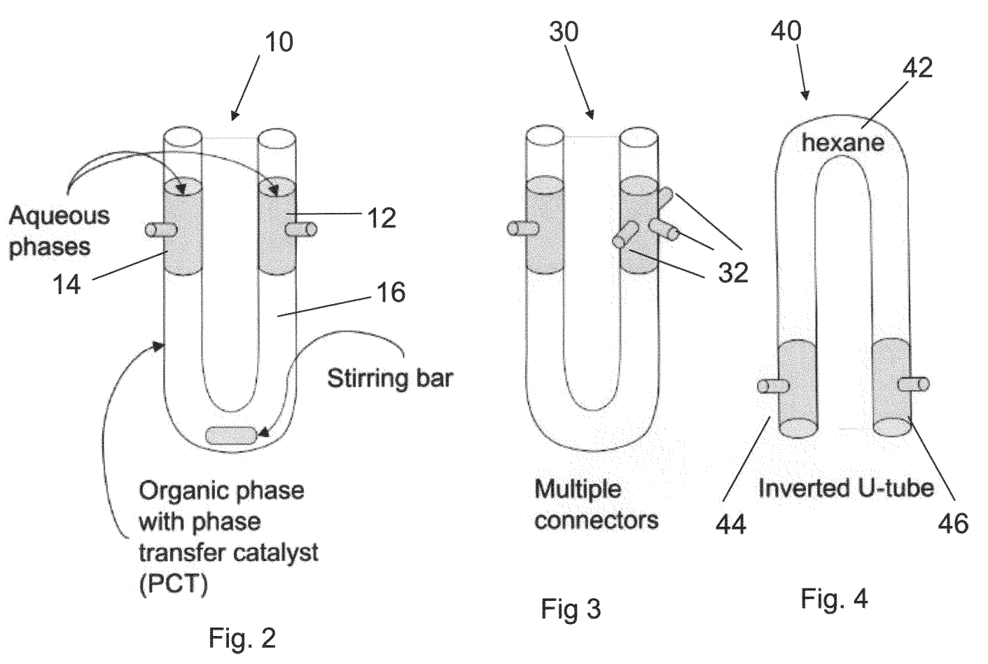

Reference is now made to FIG. 2 which illustrates a U-tube 10 in which two aqueous phases 12 and 14 are separated by an organic phase 16. The organic phase includes a phase transfer catalyst (PCT). A liquid membrane is thus formed as a computing element, from a U-tube, that holds two immiscible liquids. The more dense solution at the bottom of the tube serves as a membrane that separates into two less dense liquid phases on top of the device. One of the benefits of using a liquid membrane is that they may be highly selective. In addition, the carrier for the transport mechanism, also known as a phase transfer catalyst (PTC), is capable of specific molecular recognition and specific transfer of the recognized molecule. A liquid membrane according to the present embodiments may thus have industrial application.

There are four basic types of transport systems, cation transport, anion transport, neutral molecule transport and switchable transport, each of which has its own mechanisms and PTC types. Each system may include soluble individual molecules or aggregates of molecules and even gaseous materials, such as carbon dioxide, hydrogen, oxygen, and ammonia, as either comuting components or transporting agents. Furthermore, various PTCs can be used in the organic phase, including hydrophobic cations, such as quaternary ammonium salt, hydrophobic anions, such as tetraphenylborate, crown ethers and other binding molecules. Photochemistry and electrochemistry can also be used to increase the rates at which the carrier complexes dissociate, which increase the transport rate.

More specifically, FIG. 2 shows bulk liquid membrane device 10 in the form of a U-tube. The organic phase, such as CHCl₃ in the bottom of the tube may contain a soluble phase transfer catalyst. Two aqueous phases are placed in the arms of the U-tube, floating on top of the organic membrane. Magnetic stirring bar 18 is located in the organic phase to provide transport energy. With the magnetic stirring bar 18 rotating at fairly slow speeds, in the range of 100 to 300 rpm, the transported amounts of materials are determined by the concentrations in the receiving phase. Stability is maintained so long as the stirrer is not spinning too quickly.

Using this setup, transport of the electrically charged, and neutral species has been demonstrated.

Regarding the setup of the experiment, reference is made to R. M. Izatt, G. A. Clark, J. S. Bradshaw, J. D. Lamb, J. J. Christensen, Macrocycle-Facilitated Transport of Ions in Liquid Membrane Systems. Separation & Purification Reviews 1986, 15, 21-72, the contents of which are hereby incorporated by reference. The article describes ion transport in various liquid membrane systems in terms of those factors which create the environment for efficient and selective transport. The following parameters which affect ion transport are discussed: membrane configuration, cation-macrocycle complex stability, macrocycle partitioning between membrane and water phases, proton ionization of acidic macrocycles, macrocycle concentration, anion type, ion concentration, membrane solvent type and receiving phase composition. A summary of existing models of ion transport is given along with possible applications to macrocycle-facilitated liquid membrane-ion transport.

Reference is now made to FIG. 3 in which U-tube 30 provides a liquid membrane device in which each aqueous layer can be connected to more than one neighboring U-tube via branch connectors 32. The branch connectors are made directly to the same phase in neighboring tubes, which are considered to form a single region.

Reference is briefly made to FIG. 4 which shows inverted U-tube 40. An inverted U-tube can be used in cases of an organic layer 42 that is less dense than the water used in aqueous phases 44 and 46. Such a lighter organic substance may, for example, be hexane.

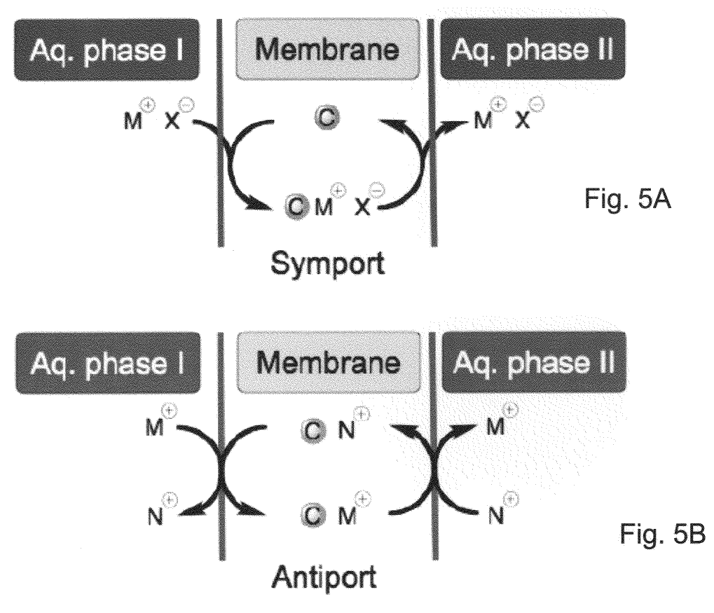

Reference is now made to FIGS. 5A and 5B which illustrate first and second modes of ion transport across the membrane. Ion transport can occur in either symport or antiport, and these are demonstrated for cations in FIGS. 5A and 5B respectively.

In the symport configuration, a neutral carrier (C) moves the guest cation together with the co-transported anion across the liquid membrane. In antiport the same carrier transports ions from phase I to phase II and different ions in the back direction from phase II to phase I.

More specifically, two modes of cation transport can be provided through a liquid membrane according to the present embodiments. In FIG. 5A, Symport transport mode is shown to occur in four stages. First, a guest salt is complexed with a carrier (C) at the interface between aqueous phase I and the membrane. Then the complex diffuses across the membrane, and the guest salt is released at the interface of the membrane with aqueous phase II. Finally, the carrier C diffuses back across the membrane, ready to start another cycle.

In the antiport mode illustrated in FIG. 5B, four stages are still present but are different. First, the anionic carrier (C) forms an ion-pair with the guest cation at the interface between aqueous phase I and the membrane, then the ion-pair diffuses across the membrane, a cation-exchange reaction releases the guest cation to phase II and finally, the carrier complex with the counter-transported ion diffuses back across the membrane. The result is bidirectional transport across the membrane.

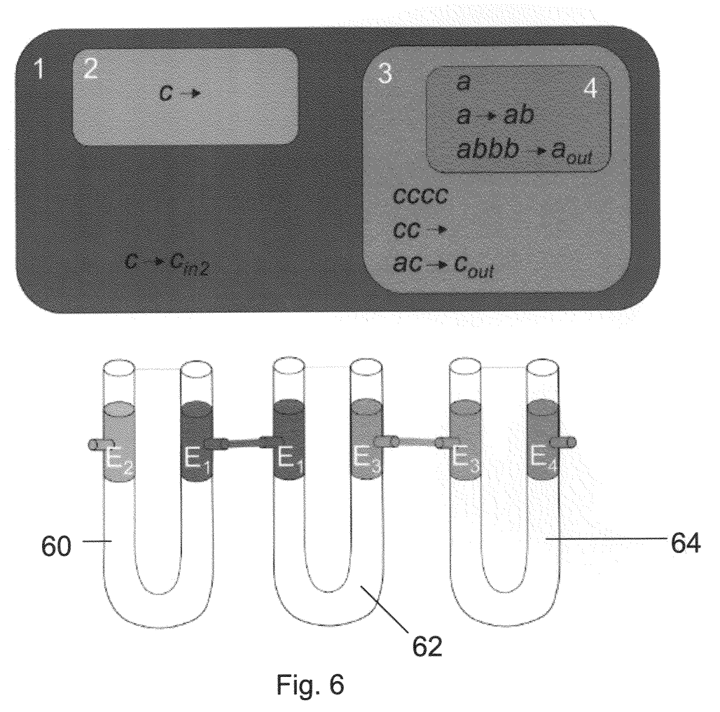

Reference is now made to FIG. 6, which illustrates how the liquid membrane devices of FIG. 2 can be connected to one another. The configuration shown in fact provides an experimental setup for realization of the P system shown in FIG. 1. The system as shown is constructed from standard, modular components.

FIG. 6 shows the scheme of FIG. 1 over an arrangement of U-tubes and numbers each region in accordance with the scheme of FIG. 1 so that it can easily be seen that the U-tube arrangement in fact represents FIG. 1, and thus provides a practical realization of a counter based on a P system, using an array of liquid membranes in interconnected U-tubes.

Three U tubes, 60, 62 and 64 are used to realize the four regions of FIG. 1. Each U-tube contains two aqueous layers floating on top of an organic phase that is denser than water, such as chloroform. Region 1 accommodates Enzyme 1. Substrate 1, which is inserted into region 1, undergoes an enzyme catalyzed reaction that converts it to substrate 2. Substrate 2 is sufficiently hydrophobic to diffuse through the organic phase to both regions 2 and 3. In each region substrate 2 is transformed differently due to the presence of different enzymes (E2 and E3, respectively).

Thus, substrate 3, which is formed from substrate 2 by enzyme 3, diffuses through the liquid membrane to region 4, and also back to region 1. In region 4 substrate 3 is transformed to substrate 5.

In principle, each substrate/product can be detected by various methods based on its properties, such as color, fluorescence, etc. Each aqueous phase can accommodate more than one enzyme and can support multiple reactions. The regions, substrates and enzymes are selected to accommodate the particular rules of any given computation.

Here is a potential cascade of 6 enzyme-catalyzed reactions where each enzyme transforms the compound produced by the previous enzyme in this cascade. Note that a branching point is provided by aldolase 1, which produces two products, acetaldehyde and pyruvic acid, each of which is further transformed by other enzymes.

| Enzyme | Reactant | Product |
|---|---|---|
| alcohol- | ethyl 2-hydroxypent-4- | ethyl 2-oxopent-4-enoate |
| dehydrogenase | enoate | |
| Lipase | ethyl 2-oxopent-4-enoate | 2-oxopent-4-enoic acid |
| Hydratase | 2-oxopent-4-enoic acid | 2-oxo-4-hydroxypentanoic |
| | | acid |
| aldolase 1 | 2-oxo-4-hydroxypentanoic | pyruvic acid + |
| | acid | acetaldehyde |
| aldolase 2 | Acetaldehyde + | 3,4-dihydroxypentan-2-one |
| | hydroxyacetone | |
| Lactate | pyruvic acid | lactic acid |
| dehydrogenase | | |

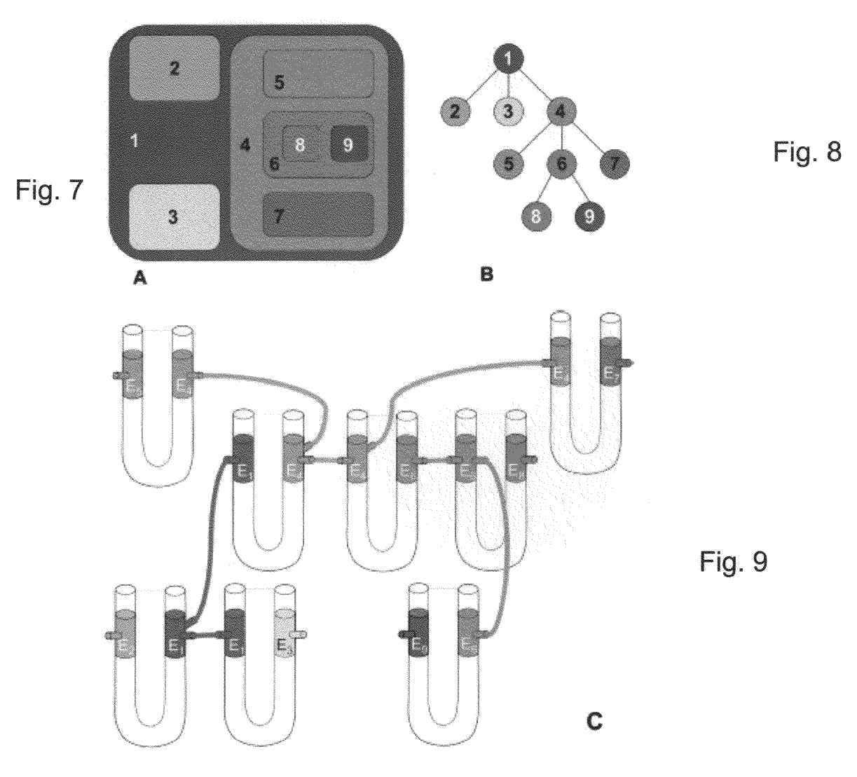

Reference is now made to FIG. 7, which illustrates a nine-region P-system. Skin region 1 surrounds eight other regions, of which it has a direct boundary with regions 2, 3 and 4. Regions 5, 6 and 7 are entirely included in region 4 and regions 8 and 9 are in turn entirely included in region 6. FIG. 8 illustrates the interrelationships in a tree and branch diagram.

FIG. 9 illustrates an arrangement of U-tubes which provides a practical implementation of the P-system of FIG. 7. FIG. 9 makes use of a mixture of the U-tubes shown in FIG. 2 with single branches and the U-tubes of FIG. 3 with multiple branches. As explained above connections via branches constitute continuations of the same phase, and only the organic phases in the U-tube constitute membrane boundaries.

More generally, each aqueous phase contains an enzyme or a mixture of several enzymes or whole-cell bacteria that can perform a catalytic task or tasks. Other biocatalysts can also be employed, such as catalytic antibodies, organic, organometallic catalysts, as well as non-catalytic reagents.

Furthermore, these aqueous reactors may accommodate photochemical, or electrochemical reactions, or any other chemical reaction. As a further variation, the orders of organic/aqueous phases can be inverted, so that the organic media accommodates the cascade of chemical reactions and the aqueous phase functions as the liquid membrane.

An advantage of the present embodiment using U-tubes is that, in contrast to living cells, all regions of the modular array are easily accessible for insertion of chemicals and other objects. Thus, input objects can be inserted directly to each U-tube in the form of substrates, inhibitors, catalysts, etc. Furthermore, detectable output can be designed to appear in a programmable way in any of the reaction compartments. Detection can be achieved by change of color, pH, fluorescence, electrochemically active compounds, chemoluminescence, change in ion concentration, for example change in Ph, change in conductivity etc.

These systems can be miniaturized using microfluidic components. Systems constructed according to the present embodiments are useful for implementation of logic gates, including Boolean components, and for construction of chemical transistors.

The terms “comprises”, “comprising”, “includes”, “including”, “having” and their conjugates mean “including but not limited to”. This term encompasses the terms “consisting of” and “consisting essentially of”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A component for computing comprising:
a first liquid phase,
a liquid separation, and
a second liquid phase separated from said first liquid phase by said liquid separation, at least one of said liquid phases initially comprising a reaction substrate and a reaction rule and said liquid separation comprising a transport rule, such as to allow reactions and transportation to occur in said component, a computing result being obtained from at least one of substrate identification and location following said reactions and transportation.

2. The component of claim 1, wherein said liquid and separation phases are retained by mutual immiscibility of the respective liquids.

3. The component of claim 1, wherein said transport rule is provided by a phase transfer catalyst.

4. The component of claim 1, wherein said reaction rule is provided by one member of the group comprising an enzyme, a mixture of enzymes, a biocatalyst, a catalytic antibody, an organic catalyst, an organometallic catalyst, and a reagent.

5. The component of claim 1, wherein transport is assisted by one member of the group comprising a mixing device located within the liquid separation, photochemical activity and electrochemical activity.

6. The component of claim 1, wherein said liquid component comprises a transport mechanism being any one of the group comprising cation transport, anion transport, neutral molecule transport and switchable transport.

7. The component of claim 1, wherein detection of said computing result comprises making use of any one of the group comprising: change of color, colorimetry, pH, fluorescence, electrochemically active compounds, chemoluminescence, change in ion concentration, change in Ph, and change in conductivity.

8. The component of claim 1, comprising branches for connecting each liquid phase to that of a neighboring component, thereby to allow construction of a component array.

9. A method of implementation of a P-system in membrane computing comprising:
placing three mutually immiscible liquids into a container, such that a first of said liquids provides a barrier between the second and third liquids;
providing an initial substrate to at least one of said second and third liquids; and
applying a transport rule for said first liquid to allow transport of substrate between said second and said third liquid.

10. The method of claim 9, comprising providing said transport rule by insertion of a phase transfer catalyst into said first liquid.

11. The method of claim 10, further comprising providing a reaction rule in at least one of said second and third liquids.

12. The method of claim 11, comprising providing said reaction rule by addition of at least one member of the group comprising an enzyme, a mixture of enzymes, a biocatalyst, a catalytic antibody, an organic catalyst, an organometallic catalyst, and a reagent, said member selected to catalyze a reaction on a substrate.

13. The method of claim 12 comprising adding substrates, phase transfer catalysts and enzymes to said respective liquids to provide initial conditions for a predetermined computing function.

14. The method of claim 13 comprising combining a plurality of said containers into an array, such that said liquids define a plurality of reaction regions separated by a plurality of barriers.

15. The method of claim 14, wherein at least one of said barriers provides symport transport.

16. The method of claim 14, wherein at least one of said barriers provides antiport transport.

17. The method of claim 14, wherein said second and third liquids are aqueous, said first liquid is non-aqueous, and a substrate is hydrophobic.

18. The method of claim 14, wherein said second and third liquids are non-aqueous, said first liquid is aqueous, and a substrate is hydrophillic.

19. The method of claim 11, further comprising detecting a computing result by making use of any one of the group comprising: change of color, colorimetry, pH, fluorescence, electrochemically active compounds, chemoluminescence, change in ion concentration, for example change in Ph, and change in conductivity.

20. The method of claim 10, wherein said second and third liquids are aqueous, said first liquid is non-aqueous and said phase transfer catalyst is hydrophobic.
